(19) United States
(12) Patent Application Publication
(10) Pub. No.: US 2025/0049926 A1
(43) Pub. Date: Feb. 13, 2025

(54) PHARMACEUTICAL COMPOSITIONS COMPRISING MODIFIED BETACYCLODEXTRINS

(71) Applicant: SILLAJEN, INC., Seoul (KR)

(72) Inventors: Luc BURY, Allschwil (CH); Markus HEUBES, Allschwil (CH); Gerrit HAUCK, Allschwil (CH)

(21) Appl. No.: 18/717,589

(22) PCT Filed: Dec. 14, 2022
(86) PCT No.: PCT/KR2022/020393

§ 371 Date: Jun. 7, 2024

(30) Foreign Application Priority Data
Dec. 15, 2021 (EP) 21214940.5
Dec. 15, 2021 (EP) 21214941.3
Jun. 28, 2022 (EP) 22181675.4
Aug. 22, 2022 (EP) 22191429.4

(51) Int. Cl. A61K 47/40; A61K 9/00; A61K 31/519

(52) U.S. Cl. CPC A61K 47/40; A61K 9/0019; A61K 31/519

(57) ABSTRACT

The present invention relates to pharmaceutical compositions comprising a compound of formula (I) as defined in the claims or a pharmaceutically acceptable salt thereof and a modified beta-cyclodextrin, as well as methods of using the pharmaceutical compositions for the treatment of neoplastic diseases such as cancer.

### TECHNICAL FIELD

The present invention relates to pharmaceutical compositions comprising the active pharmaceutical compound described below and modified beta-cyclodextrin excipients, as well as methods of using the pharmaceutical compositions for the treatment of neoplastic diseases, in particular cancer.

### BACKGROUND ART

WO 2015/155042 describes a recently discovered class of inhibitors of the threonine tyrosine kinase (TTK) for use in the treatment of cancer.

Cyclodextrins are cyclic oligosaccharides containing six (alpha-cyclodextrins), seven (beta-cyclodextrins) or eight (gamma-cyclodextrins) glucose units linked via alpha-1,4-glycosidic bonds. Derivatized versions of cyclodextrins are used as pharmaceutical excipients.

### DISCLOSURE OF INVENTION

### Solution to Problem

### Summary of the Invention

In a first aspect the present invention provides pharmaceutical compositions comprising a compound of formula (I)

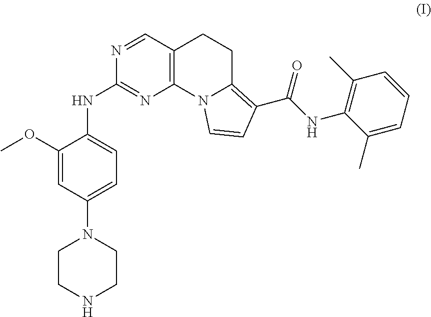 [SMILES: COc1cc(N2CCNCC2)ccc1Nc1ncc2c(n1)-n1ccc(C(=O)Nc3c(C)cccc3C)c1CC2]

or a pharmaceutically acceptable salt thereof and a modified beta-cyclodextrin.

As a dual TTK/polo-like kinase 1 (PLK1) inhibitor, the compound of formula (I) represents a first-in-class in an emerging class of next generation mitotic therapeutics and as far as the inventors are aware it is the only TTK inhibitor to cause regressions (and cures) when administered as a single agent in mouse tumor models. However the compound of formula (I) has low aqueous solubility, leading to the possibility of limited exposure in humans.

For phase I studies the inventors aimed to develop a formulation able to deliver up to 500 mg of the compound of formula (I) as an intravenous infusion over 30 to 60 minutes taking into account the poor solubility of the compound of formula (I) and the maximum accepted daily intake (ADI) of excipients used to improve active pharmaceutical ingredient (API) solubilization. It has surprisingly been found that sulfobutyl ether-beta-cyclodextrin provides acceptable solubilization of the compound of formula (I) without exceeding its maximum ADI.

In a further aspect, the invention provides methods of treating neoplastic diseases such as cancer in a patient in need thereof comprising administering the pharmaceutical composition of the invention, e.g. in a therapeutically effective amount, to said patient.

In a further aspect, the invention provides the pharmaceutical compositions of the invention for use in treating neoplastic diseases such as cancer in a patient.

In a further aspect, the invention provides use of the pharmaceutical compositions of the invention in the manufacture of medicaments for the treatment of neoplastic diseases such as cancer in a patient.

Additional aspects and embodiments of the invention are described in more detail below.

### DETAILED DESCRIPTION OF THE INVENTION

### General Definitions

Certain terms used herein are described below. Unless defined otherwise, all technical and scientific terms used herein have the same meaning as is commonly understood by one of skill in the art to which this invention belongs.

The term “pharmaceutical composition” is defined herein to refer to a liquid formulation containing at least one therapeutic agent to be administered to a patient with one or more pharmaceutically acceptable excipients, in order to prevent or treat a particular disease or condition affecting the patient.

The term “pharmaceutically acceptable” as used herein refers to items such as compounds, materials, compositions and/or dosage forms, which are, within the scope of sound medical judgment, suitable for contact with the tissues of a warm-blooded animal, e.g., a mammal or human, without excessive toxicity or other complications commensurate with a reasonable benefit/risk ratio.

The term “treatment,” as used herein in the context of treating a neoplastic disease in a patient pertains generally to treatment and therapy in which some desired therapeutic effect is achieved, for example, the inhibition of the progress of the neoplastic disease, and includes a reduction in the rate of progress, a halt in the rate of progress, alleviation of symptoms of the neoplastic disease, amelioration of neoplastic disease, and cure of the neoplastic disease. For example, treatment can be the diminishment of one or several symptoms of a disorder or complete eradication of a disorder, such as cancer. Within the meaning of the present disclosure, the term “treat” also denotes to arrest, delay the onset (i.e. the period prior to clinical manifestation of a disease) and/or reduce the risk of developing or worsening a disease.

The term “pharmaceutically effective amount”, “therapeutically effective amount” or “clinically effective amount” is an amount sufficient to provide an observable or clinically significant improvement over the baseline clinically observable signs and symptoms of the disorders treated with the pharmaceutical composition.

The term “patient” refers to a human presenting themselves for therapeutic treatment.

The term “about” means a variation of no more than 10% of the relevant figure. In some embodiments, the term “about” means a variation of no more than 5% of the relevant figure.

Where ranges are given the end points of the range are included in the range.

### Compound of Formula (I)

Reference to the compound of formula (I) refers to the free base and pharmaceutically acceptable salts of the compound of formula (I) depicted above. In some embodiments reference to the compound of formula (I) refers to the free base of the compound of formula (I). In other embodiments reference to the compound of formula (I) refers to a pharmaceutically acceptable salt of the compound of formula (I).

Pharmaceutically acceptable salts of the compound of formula (I) may be acid addition salts. Salts are formed e.g. with organic or inorganic acids from compounds of formula (I). Pharmaceutically acceptable salts are within the common general knowledge of the person skilled in the art. Pharmaceutically acceptable salts may include more than one molecule or ion of the corresponding acid.

Compounds of formula (I) and pharmaceutically acceptable salts thereof may be synthesized as described in WO 2015/155042, in particular on pages 17 to 19 which are hereby incorporated by reference, and as described in Example 17 on page 49 of WO 2015/155042, which is also hereby incorporated by reference, including the reference in Example 17 to Example 9, Intermediate H and Example 1.

In some embodiments the concentration of the compound of formula (I) or a pharmaceutically acceptable salt thereof in the pharmaceutical composition of the invention is about 1 mg/mL to about 3.5 mg/mL based on the compound of formula (I) as free base. In some embodiments, the concentration of the compound of formula (I) or a pharmaceutically acceptable salt thereof in the pharmaceutical composition of the invention is about 1 mg/mL to about 3 mg/mL based on the compound of formula (I) as free base. In some embodiments, the concentration of the compound of formula (I) or a pharmaceutically acceptable salt thereof in the pharmaceutical composition of the invention is about 1.5 mg/mL to about 2.5 mg/mL based on the compound of formula (I) as free base.

In some embodiments, the concentration of the compound of formula (I) or a pharmaceutically acceptable salt thereof in the pharmaceutical composition of the invention is up to about 3.5 mg/mL based on the compound of formula (I) as free base. In some embodiments, the concentration of the compound of formula (I) or a pharmaceutically acceptable salt thereof in the pharmaceutical composition of the invention is up to about 3 mg/mL based on the compound of formula (I) as free base. In some embodiments, the concentration of the compound of formula (I) or a pharmaceutically acceptable salt thereof in the pharmaceutical composition of the invention is up to about 2.5 mg/mL based on the compound of formula (I) as free base.

In some embodiments, the concentration of the compound of formula (I) or a pharmaceutically acceptable salt thereof in the pharmaceutical composition of the invention is at least about 1 mg/mL based on the compound of formula (I) as free base. In some embodiments, the concentration of the compound of formula (I) or a pharmaceutically acceptable salt thereof in the pharmaceutical composition of the invention is at least about 1.5 mg/mL based on the compound of formula (I) as free base.

In some embodiments, the concentration of the compound of formula (I) or a pharmaceutically acceptable salt thereof in the pharmaceutical composition of the invention is about 2 mg/mL based on the compound of formula (I) as free base.

Generally, higher solubilized concentrations of the compound of formula (I) in the pharmaceutical compositions of the invention can be achieved at lower pH.

The expression “based on the compound of formula (I) as free base” means that when a pharmaceutically acceptable salt of the compound of formula (I) is used, the amount in mg/mL of the pharmaceutically acceptable salt of the compound of formula (I) is the mole equivalent of the stated amount in mg/mL of the free base of the compound of formula (I).

### Modified Beta-Cyclodextrins

Cyclodextrins interact with hydrophobic drug molecules to form inclusion complexes. beta-Cyclodextrin has a relatively low aqueous solubility and modification of the beta-cyclodextrin via substitution of any of the hydrogen bond-forming hydroxyl groups, even by lipophilic substituents, results in a dramatic improvement in the aqueous solubility of the derivative. Hydrophobic, hydrophilic, polymerized, ionized, non-ionized and many other derivatives of cyclodextrins have been developed and substituents include neutral, anionic and/or cationic functional groups.

Modified cyclodextrin, e.g. alkylated cyclodextrins, include for example sulfoalkyl ether cyclodextrins, alkyl ether cyclodextrins (e.g. methyl, ethyl and propyl ether cyclodextrins), hydroxyalkyl cyclodextrins, thioalkyl ether cyclodextrins, carboxylated cyclodextrins (e.g. succinyl-beta-cyclodextrin etc.), sulfated cyclodextrins etc. Alkylated cyclodextrins having more than one type of functional group are also known, such as sulfoalkyl ether-alkyl ether-cyclodextrins (see, e.g. WO 2005/042584 and US 2009/0012042).

Particular examples of modified beta-cyclodextrin derivatives used as excipients in medicines are the sulfobutyl ether derivative of beta-cyclodextrin (SBE-beta-CD), the hydroxypropyl derivative of beta-cyclodextrin (HP-beta-CD), and the randomly methylated beta-cyclodextrin (RM-beta-CD).

The structure of modified beta-cyclodextrin is shown below (formula II), wherein each R independently represents hydrogen or an aliphatic substituent, wherein at least one R is not hydrogen.

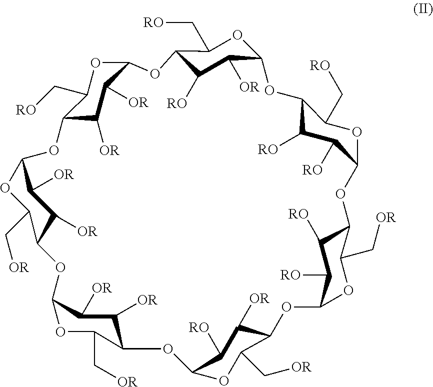

In SBE-beta-CD R is —(CH₂)₄—SO₃Na, in HP-beta-CD R is —CH₂—CHOH—CH₃, in RM-beta-CD R is —CH₃ (see e.g. Cyclodextrins used as excipients, EMA/CHMP/333892/2013)—note that in each case R is a mixture of the given substituent and hydrogen. For a further review of the use of cyclodextrins in pharmaceuticals, in particular in parenteral formulations, see Loftsson, Journal of Pharmaceutical Sciences 2021, 110:654-664.

SBE-beta-CD has the CAS number 182410-00-0, and for example is sold under the tradenames Captisol™ (Ligand) and Dexolve™ (Cyclolab). It is listed in both the European and US Pharmacopoeia. Captisol™ has a molecular weight of 2163 g/mol based on an average degree of substitution of 6.5 (www.captisol.com). According to U.S. Pat. No. 9,493,582 the degree of substitution in Captisol™ is 6 to 7.1. Dexolve™ also has an average degree of substitution of 6.5 (www.cyclolab.hu). SBE-beta-CDs can be manufactured as described for example in patent applications US 2009/011037, US 2009/270348, US 2015/045311, US 2015/284479 and US 2016/009826, all of which are incorporated by reference.

In some embodiments, the modified beta-cyclodextrin (e.g. SBE-beta-CD) has a degree of substitution of about 2 to about 9. In some embodiments, the modified beta-cyclodextrin (e.g. SBE-beta-CD) has a degree of substitution of about 4.5 to about 7.5. In some embodiments, the modified beta-cyclodextrin (e.g. SBE-beta-CD) has a degree of substitution of about 6 to about 7.5. In some embodiments, the modified beta-cyclodextrin (e.g. SBE-beta-CD) has a degree of substitution of about 6.5.

In some embodiments, the modified beta-cyclodextrin (e.g. SBE-beta-CD) has an average degree of substitution of about 2 to about 9. In some embodiments the modified beta-cyclodextrin (e.g. SBE-beta-CD) has an average degree of substitution of about 4.5 to about 7.5. In some embodiments, the modified beta-cyclodextrin (e.g. SBE-beta-CD) has an average degree of substitution of about 6 to about 7.5. In some embodiments, the modified beta-cyclodextrin (e.g. SBE-beta-CD) has an average degree of substitution of about 6.5. In some embodiments, the modified beta-cyclodextrin is SBE-beta-CD having the CAS number 182410-00-0.

The degree of substitution is a measure of the number of substituents attached to the modified beta-cyclodextrin molecule in term of moles of substituent per mole of modified beta-cyclodextrin. The average degree of substitution is a measure of the total number of substituents present per modified beta-cyclodextrin molecule for the distribution of the modified beta-cyclodextrins within the modified beta-cyclodextrin product used in the pharmaceutical composition of the invention.

The amount of modified beta-cyclodextrin (e.g. SBE-beta-CD) used in the pharmaceutical compositions of the invention should be sufficient to dissolve the compound of formula (I) or pharmaceutically acceptable salt thereof. In some embodiments, the concentration of modified beta-cyclodextrin (e.g. SBE-beta-CD) in the pharmaceutical composition of the invention is about 10 mg/mL to about 400 mg/mL. In some embodiments, the concentration of modified beta-cyclodextrin (e.g. SBE-beta-CD) in the pharmaceutical composition of the invention is about 10 mg/mL to about 200 mg/mL. In some embodiments, the concentration of modified beta-cyclodextrin (e.g. SBE-beta-CD) in the pharmaceutical composition of the invention is about 10 mg/mL to about 100 mg/mL. In some embodiments, the concentration of modified beta-cyclodextrin (e.g. SBE-beta-CD) in the pharmaceutical composition of the invention is about 20 mg/mL to about 60 mg/mL. In some embodiments, the concentration of modified beta-cyclodextrin (e.g. SBE-beta-CD) in the pharmaceutical composition of the invention is about 30 mg/mL to about 50 mg/mL.

In some embodiments, the concentration of modified beta-cyclodextrin (e.g. SBE-beta-CD) in the pharmaceutical composition of the invention is up to about 400 mg/mL. In some embodiments, the concentration of modified beta-cyclodextrin (e.g. SBE-beta-CD) in the pharmaceutical composition of the invention is up to about 200 mg/mL. In some embodiments, the concentration of modified beta-cyclodextrin (e.g. SBE-beta-CD) in the pharmaceutical composition of the invention is up to about 100 mg/mL. In some embodiments, the concentration of modified beta-cyclodextrin (e.g. SBE-beta-CD) in the pharmaceutical composition of the invention is up to about 60 mg/mL. In some embodiments, the concentration of modified beta-cyclodextrin (e.g. SBE-beta-CD) in the pharmaceutical composition of the invention is up to about 50 mg/mL.

In some embodiments, the concentration of modified beta-cyclodextrin (e.g. SBE-beta-CD) in the pharmaceutical composition of the invention is at least about 10 mg/mL. In some embodiments, the concentration of modified beta-cyclodextrin (e.g. SBE-beta-CD) in the pharmaceutical composition of the invention is at least about 20 mg/mL. In some embodiments, the concentration of modified beta-cyclodextrin (e.g. SBE-beta-CD) in the pharmaceutical composition of the invention is at least about 30 mg/mL.

In some embodiments, the concentration of modified beta-cyclodextrin (e.g. SBE-beta-CD) in the pharmaceutical composition of the invention about 40 mg/mL.

### pH, Additives, Compositions

As mentioned above, the pH of the composition has an impact on the solubility of the compounds of formula (I) in SBE-beta-CD, with solubility increasing at lower pH.

In some embodiments the pH of the pharmaceutical composition of the invention is about 3.5 to about 9, bearing in mind that higher pH values correspond to lower solubility of the compound of formula (I). In some embodiments, the pH of the pharmaceutical composition of the invention is about 3.5 to about 7. In some embodiments, the pH of the pharmaceutical composition of the invention is about 4 to about 6.5. In some embodiments, the pH of the pharmaceutical composition of the invention is about 4.5 to about 6.5. In some embodiments, the pH of the pharmaceutical composition of the invention is 4.5 to 6.5. In some embodiments, the pH of the pharmaceutical composition of the invention is about 4.5 to about 5.5.

In some embodiments, the pH of the pharmaceutical composition of the invention is at least about 3.5. In some embodiments, the pH of the pharmaceutical composition of the invention is at least about 4. In some embodiments, the pH of the pharmaceutical composition of the invention is at least about 4.5. In some embodiments, the pH of the pharmaceutical composition of the invention is at least 4.5.

In some embodiments, the pH of the pharmaceutical composition of the invention is up to about 9. In some embodiments, the pH of the pharmaceutical composition of the invention is up to about 7. In some embodiments, the pH of the pharmaceutical composition of the invention is up to about 6.5. In some embodiments, the pH of the pharmaceutical composition of the invention is up to 6.5. In some embodiments, the pH of the pharmaceutical composition of the invention is up to about 5.5.

In some embodiments, the pH of the pharmaceutical composition of the invention is about 5.5.

The pH of the pharmaceutical composition of the invention may be maintained by a suitable buffering agent. Examples include citrate, acetate, gluconate, lactate and aspartate. The person skilled in the art will be aware of many other suitable buffers which could be used with the pharmaceutical compositions of the invention in order to control the pH.

The buffering agent may be used at a concentration as determined as suitable by a person skilled in the art. Concentrations such as about 5 mM to about 100 mM may be used, e.g. about 10 mM to about 100 mM, e.g. about 20 mM to about 50 mM, e.g. up to about 100 mM, e.g. up to about 50 mM, e.g. at least about 10 mM, e.g. at least about 20 mM, e.g. about 30 mM.

The pharmaceutical compositions may include an osmolality agent such as a salt, e.g. sodium chloride, in addition to buffer. The salt such as sodium chloride may be used at a concentration as determined as suitable by a person skilled in the art. In some embodiments, a salt may be omitted. When a salt is present, concentrations such as about 0.5 mg/mL to about 10 mg/mL may be used, e.g. about 0.5 mg/mL to about 8 mg/mL, e.g. about 1 mg/mL to about 8 mg/mL, e.g. about 2 mg/mL to about 6 mg/mL, e.g. up to about 10 mg/mL, e.g. up to about 8 mg/mL, e.g. up to about 6 mg/mL, e.g. at least about 0.5 mg/mL, e.g. at least about 1 mg/mL e.g. at least about 2 mg/ml e.g. at least about 3 mg/mL, e.g. about 3 mg/mL.

Other additives may be used in the pharmaceutical compositions of the invention, e.g. antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as Tween™ (e.g. Tween™ 80), Pluronics™, or polyethylene glycol (PEG); as well as the co-solvents, hydrophilic polymers and surfactants as described in the Examples below.
In some embodiments, the pharmaceutical composition comprises the compound of formula (I) or a pharmaceutically acceptable salt thereof, and SBE-beta-CD (e.g. having an average degree of substitution of about 6 to about 7.5), wherein the pH of the pharmaceutical composition is about 4 to about 6.5.

In some embodiments, the pharmaceutical composition comprises the compound of formula (I) or a pharmaceutically acceptable salt thereof at a concentration of about 1 mg/mL to about 3 mg/mL based on the compound of formula (I) as free base, and SBE-beta-CD (e.g. having an average degree of substitution of about 6 to about 7.5), wherein the pH of the pharmaceutical composition is about 4 to about 6.5.

In some embodiments, the pharmaceutical composition comprises the compound of formula (I) or a pharmaceutically acceptable salt thereof at a concentration of about 1.5 mg/mL to about 2.5 mg/mL based on the compound of formula (I) as free base, and SBE-beta-CD (e.g. having an average degree of substitution of about 6 to about 7.5), wherein the pH of the pharmaceutical composition is about 4 to about 6.5.

In some embodiments, the pharmaceutical composition comprises the compound of formula (I) or a pharmaceutically acceptable salt thereof, and SBE-beta-CD (e.g. having an average degree of substitution of about 6 to about 7.5) at a concentration of about 10 mg/mL to about 200 mg/mL, wherein the pH of the pharmaceutical composition is about 4 to about 6.5.

In some embodiments, the pharmaceutical composition comprises the compound of formula (I) or a pharmaceutically acceptable salt thereof, and SBE-beta-CD (e.g. having an average degree of substitution of about 6 to about 7.5) at a concentration of about 10 mg/mL to about 100 mg/mL, wherein the pH of the pharmaceutical composition is about 4 to about 6.5.

In some embodiments, the pharmaceutical composition comprises the compound of formula (I) or a pharmaceutically acceptable salt thereof, and SBE-beta-CD (e.g. having an average degree of substitution of about 6 to about 7.5) at a concentration of about 30 mg/mL to about 50 mg/mL, wherein the pH of the pharmaceutical composition is about 4 to about 6.5.

In some embodiments, the pharmaceutical composition comprises the compound of formula (I) or a pharmaceutically acceptable salt thereof at a concentration of about 1 mg/mL to about 3 mg/mL based on the compound of formula (I) as free base, and SBE-beta-CD (e.g. having an average degree of substitution of about 6 to about 7.5) at a concentration of about 10 mg/mL to about 200 mg/mL, wherein the pH of the pharmaceutical composition is about 4 to about 6.5.

In some embodiments, the pharmaceutical composition comprises the compound of formula (I) or a pharmaceutically acceptable salt thereof at a concentration of about 1 mg/mL to about 3 mg/mL based on the compound of formula (I) as free base, and SBE-beta-CD (e.g. having an average degree of substitution of about 6 to about 7.5) at a concentration of about 10 mg/mL to about 100 mg/mL, wherein the pH of the pharmaceutical composition is about 4 to about 6.5.

In some embodiments, the pharmaceutical composition comprises the compound of formula (I) or a pharmaceutically acceptable salt thereof at a concentration of about 1.5 mg/mL to about 2.5 mg/mL based on the compound of formula (I) as free base, and SBE-beta-CD (e.g. having an average degree of substitution of about 6 to about 7.5) at a concentration of about 30 mg/mL to about 50 mg/mL, wherein the pH of the pharmaceutical composition is about 4 to about 6.5.

In some embodiments, the pharmaceutical composition comprises the compound of formula (I) or a pharmaceutically acceptable salt thereof at a concentration of about 2 mg/mL based on the compound of formula (I) as free base, and SBE-beta-CD (e.g. having an average degree of substitution of about 6 to about 7.5), at a concentration of about 40 mg/mL, wherein the pH of the pharmaceutical composition is 4.5 to 6.5 (e.g. about 5.5).

In some embodiments, the pharmaceutical composition consists essentially of the compound of formula (I) as free base, SBE-beta-CD (e.g. having an average degree of substitution of about 6 to about 7.5), one or more buffering agents, optionally a salt, and optionally water, wherein the pH of the pharmaceutical composition is about 4 to about 6.5.

In some embodiments, the pharmaceutical composition consists essentially of the compound of formula (I) as free base at a concentration of about 1 mg/mL to about 3 mg/mL, SBE-beta-CD (e.g. having an average degree of substitution of about 6 to about 7.5), one or more buffering agents, optionally a salt, and optionally water, wherein the pH of the pharmaceutical composition is about 4 to about 6.5.

In some embodiments, the pharmaceutical composition consists essentially of the compound of formula (I) as free base at a concentration of about 1.5 mg/mL to about 2.5 mg, SBE-beta-CD (e.g. having an average degree of substitution of about 6 to about 7.5), one or more buffering agents, optionally a salt, and optionally water, wherein the pH of the pharmaceutical composition is about 4 to about 6.5.

In some embodiments, the pharmaceutical composition consists essentially of the compound of formula (I) as free base, SBE-beta-CD (e.g. having an average degree of substitution of about 6 to about 7.5) at a concentration of about 10 mg/mL to about 200 mg/mL, one or more buffering agents, optionally a salt, and optionally water, wherein the pH of the pharmaceutical composition is about 4 to about 6.5.

In some embodiments, the pharmaceutical composition consists essentially of the compound of formula (I) as free base, SBE-beta-CD (e.g. having an average degree of substitution of about 6 to about 7.5) at a concentration of about 10 mg/mL to about 100 mg/mL, one or more buffering agents, optionally a salt, and optionally water, wherein the pH of the pharmaceutical composition is about 4 to about 6.5.

In some embodiments, the pharmaceutical composition consists essentially of the compound of formula (I) as free base, SBE-beta-CD (e.g. having an average degree of substitution of about 6 to about 7.5) at a concentration of about 30 mg/mL to about 50 mg/mL, one or more buffering agents, optionally a salt, and optionally water, wherein the pH of the pharmaceutical composition is about 4 to about 6.5.

In some embodiments, the pharmaceutical composition consists essentially of the compound of formula (I) as free base at a concentration of about 1 mg/mL to about 3 mg/mL, SBE-beta-CD (e.g. having an average degree of substitution of about 6 to about 7.5) at a concentration of about 10 mg/mL to about 200 mg/mL, one or more buffering agents, optionally a salt, and optionally water, wherein the pH of the pharmaceutical composition is about 4 to about 6.5.

In some embodiments, the pharmaceutical composition consists essentially of the compound of formula (I) as free base at a concentration of about 1 mg/mL to about 3 mg/mL, SBE-beta-CD (e.g. having an average degree of substitution of about 6 to about 7.5) at a concentration of about 10 mg/mL to about 100 mg/mL, one or more buffering agents, optionally a salt, and optionally water, wherein the pH of the pharmaceutical composition is about 4 to about 6.5.

In some embodiments, the pharmaceutical composition consists essentially of the compound of formula (I) as free base at a concentration of about 1.5 mg/mL to about 2.5 mg/mL, SBE-beta-CD (e.g. having an average degree of substitution of about 6 to about 7.5) at a concentration of about 30 mg/mL to about 50 mg/mL, one or more buffering agents, optionally a salt, and optionally water, wherein the pH of the pharmaceutical composition is about 4 to about 6.5.

In some embodiments, the pharmaceutical composition consists essentially of the compound of formula (I) as free base at a concentration of about 2 mg/mL, SBE-beta-CD (e.g. having an average degree of substitution of about 6 to about 7.5) at a concentration of about 40 mg/mL, one or more buffering agents, optionally a salt, and optionally water, wherein the pH of the pharmaceutical composition is 4.5 to 6.5 (e.g. about 5.5).

In some embodiments, the pharmaceutical composition is one wherein the components and the quantities of each component per mL of pharmaceutical composition are as follows:

| Component | Quantity |
|---|---|
| compound of formula (I) as free base | about 1 mg to about 3 mg |
| SBE-β-CD (e.g. average degree of | about 10 mg to about 200 mg |
| substitution of about 6 to about 7.5) | |
| suitable buffer (e.g. citrate buffer) | about 5 mM to about 100 mM |
| salt (e.g. sodium chloride) | 0 mg/mL to about 10 mg/mL |
| sodium hydroxide | quantity sufficient to adjust |
| | the pH to about 4 to about 6.5 |
| hydrochloric acid | quantity sufficient to adjust |
| | the pH to about 4 to about 6.5 |
| water for injection | q.s. to 1 mL |
| Total Volume | 1 mL |

In some embodiments the pharmaceutical composition is one wherein the components and the quantities of each component per mL of pharmaceutical composition are as follows:

| Component | Quantity |
|---|---|
| compound of formula (I) as free base | about 1 mg to about 3 mg |
| SBE-β-CD (e.g. average degree of | about 10 mg to about 100 mg |
| substitution of about 6 to about 7.5) | |
| suitable buffer (e.g. citrate buffer) | about 5 mM to about 100 mM |
| salt (e.g. sodium chloride) | 0 mg/mL to about 10 mg/mL |
| sodium hydroxide | quantity sufficient to adjust |
| | the pH to about 4 to about 6.5 |
| hydrochloric acid | quantity sufficient to adjust |
| | the pH to about 4 to about 6.5 |
| water for injection | q.s. to 1 mL |
| Total Volume | 1 mL |

In some embodiments, the pharmaceutical composition is one wherein the components and the quantities of each component per mL of pharmaceutical composition are as follows:

| Component | Quantity |
|---|---|
| compound of formula (I) as free base | about 1.5 mg to about 2.5 mg |
| SBE-β-CD (e.g. average degree of | about 30 mg to about 50 mg |
| substitution of about 6 to about 7.5) | |
| suitable buffer (e.g. citrate buffer) | about 10 mM to about 50 mM |
| salt (e.g. sodium chloride) | 0.5 mg/mL to about 8 mg/mL |
| sodium hydroxide | quantity sufficient to adjust |
| | the pH to about 4 to about 6.5 |
| hydrochloric acid | quantity sufficient to adjust |
| | the pH to about 4 to about 6.5 |
| water for injection | q.s. to 1 mL |
| Total Volume | 1 mL |

In some embodiments, the pharmaceutical composition is one wherein the components and the quantities of each component per mL of pharmaceutical composition are as follows:

| Component | Quantity |
|---|---|
| compound of formula (I) as free base | about 2 mg |
| SBE-β-CD, average degree of | about 40 mg |
| substitution of about 6 to about 7.5 | |
| citrate buffer | about 30 mM |
| sodium chloride | about 3 mg/mL |
| sodium hydroxide | quantity sufficient to adjust |
| | the pH to 4.5 to 6.5 |
| hydrochloric acid | quantity sufficient to adjust |
| | the pH to 4.5 to 6.5 |
| water for injection | q.s. to 1 mL |
| Total Volume | 1 mL |

An example pharmaceutical composition is one wherein the components and the quantities of each component per mL of pharmaceutical composition are as follows:

| Component | Quantity |
|---|---|
| compound of formula (I) | about 2.0 mg |
| hydrochloric acid | about 3.9 mg |
| citric acid, monohydrate | about 6.3 mg |
| sodium chloride | about 3.0 mg |
| SBE-β-CD, average degree of | about 40 mg |
| substitution of about 6 to about 7.5 | |
| sodium hydroxide | q.s. to |
| | pH 4.5 to 6.5 |
| water for injection | q.s. to 1 mL |
| Total Volume | 1 mL |

### Methods of Manufacture

Compounds of formula (I) and pharmaceutically acceptable salts thereof can be prepared as described above. Modified beta-cyclodextrins can also be prepared as described above or can be obtained from commercial sources. The pharmaceutical compositions of the invention may be prepared by charging an initial volume of water (WFI) into a vessel, adding buffer and adjusting the pH to the desired level using e.g. hydrochloric acid followed by adding the compound of formula (I) or a pharmaceutically acceptable salt thereof, e.g. as a powder, and mixing until dissolved. The process may be continued by adding the modified beta-cyclodextrin and mixing until dissolved. After dissolution of the compound of formula (I) or a pharmaceutically acceptable salt thereof and modified beta-cyclodextrin the pH may be adjusted to correspond to the desired pH range and the solution made up to volume with water. The pharmaceutical composition may be sterilized by filtering using a 0.2 μm filter. Finally the pharmaceutical composition may be aliquoted into vials and sealed. The person skilled in the art will be aware that alternative methods of manufacture are also possible.

The pharmaceutical compositions of the invention can be administered intravenously to a patient without dilution or after dilution e.g. with saline solution.

### Uses of the Pharmaceutical Compositions of the Invention

The pharmaceutical compositions of the invention may be used to treat neoplastic diseases such as cancer, e.g. by intravenous administration. The pharmaceutical compositions of the invention may be used to treat a cancer at any clinical stage or pathological grade (e.g. tumor stage I, tumor stage II, tumor stage III, tumor stage IV) or treatment settings (e.g. preventative, adjuvant, neoadjuvant, therapeutic including palliative treatment). The pharmaceutical compositions of the invention may be for use in slowing, delaying or stopping cancer progression or cancer growth or increasing the overall survival time or the cancer-progression-free survival time or the time to progression of a cancer or improving or maintaining the patient's (e.g. patient's) quality of life or functional status. The pharmaceutical compositions of the invention may also be used in post-therapy recovery from cancer. The pharmaceutical compositions of the invention may be used in the treatment of metastatic cancer.

For example, the pharmaceutical compositions of the invention may be used for (i) reducing the number of cancer cells; (ii) reducing tumor volume; (iii) increasing tumor regression rate; (iv) reducing or slowing cancer cell infiltration into peripheral organs; (v) reducing or slowing tumor metastasis; (vi) reducing or inhibiting tumor growth; (vii) preventing or delaying occurrence and/or recurrence of the cancer and/or extends disease- or tumor-free survival time; (viii) increasing overall survival time; (ix) reducing the frequency of treatment; and/or (x) relieving one or more of symptoms associated with the cancer.

As mentioned above, the pharmaceutical compositions of the invention may be used for the treatment of neoplastic diseases. Examples of neoplastic diseases include, but are not limited to, epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, miscellaneous myeloproliferative disorders, lymphoproliferative disorders and myelodysplastic syndromes.

In some embodiments, the neoplastic disease is cancer. Examples of cancers in terms of the organs and parts of the body affected include, but are not limited to, the brain, breast (including triple negative breast cancer and luminal B breast cancer), cervix, ovaries, colon, rectum (including colon and rectum i.e. colorectal cancer) lung (including small cell lung cancer, non-small cell lung cancer, large cell lung cancer and mesothelioma), endocrine system, bone, adrenal gland, thymus, liver, stomach, intestine (including gastric cancer), pancreas, bone marrow, hematological malignancies (such as lymphoma, leukemia, myeloma or lymphoid malignancies), bile duct, bladder, urinary tract, kidneys, skin, thyroid, head, neck, prostate and testis.

In some embodiments, the neoplastic disease is a cancer selected from breast cancer (including triple negative breast cancer and luminal B breast cancer), gastric cancer, colorectal cancer, liver cancer (including hepatocellular cancer), endometrial cancer, ovarian cancer, esophageal cancer, lung cancer (including non-small cell lung cancer), Kaposi's sarcoma, cervical cancer, pancreatic cancer, melanoma, prostate cancer, bladder cancer and leukemia, e.g. acute myeloid leukemia (AML) (including Complex Karyotype AML).

In some embodiments, the neoplastic disease is breast cancer.

In some embodiments, the neoplastic disease is triple negative breast cancer.

In some embodiments, the neoplastic disease is luminal B breast cancer.

In some embodiments, the neoplastic disease is gastric cancer.

In some embodiments, the neoplastic disease is colorectal cancer.

In some embodiments, the neoplastic disease is hepatocellular cancer.

In some embodiments, the neoplastic disease is endometrial cancer.

In some embodiments, the neoplastic disease is acute myeloid leukemia (AML) (including Complex Karyotype AML).

In some embodiments, the neoplastic disease is lung cancer (e.g. small cell lung cancer, non-small cell lung cancer).

In some embodiments, the neoplastic disease is cervical cancer (e.g. metastatic or recurrent cervical cancer).

In some embodiments, the neoplastic disease is head and neck cancer (e.g. recurrent or metastatic squamous cell carcinoma of the head and neck).

In some embodiments, the neoplastic disease is Wilms' tumor.

In some embodiments, the neoplastic disease is a brain tumor (e.g. gliomas, such as progressive or recurrent gliomas, medulloblastoma, such as recurrent medulloblastoma).

In some embodiments, the neoplastic disease is neuroblastoma.

In some embodiments, the neoplastic disease is testicular cancer (e.g. metastatic non-seminomatous germ cell tumor).

In some embodiments, the neoplastic disease is bladder cancer (e.g. advanced bladder cancer, including those with abnormal renal function).

In some embodiments, the neoplastic disease is retinoblastoma (e.g. recurrent or progressive retinoblastoma.

The cancer may be a primary tumor and/or metastases. The cancer may be derived from a solid or liquid (e.g. hematological or intraperitoneal) tumor. In some embodiments, the neoplastic disease (e.g. cancer) to be treated is a tumor, e.g. a solid tumor.

All aspects and embodiments of the invention described herein may be combined in any combination where possible.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and should not be construed as limiting the invention in any way.

### Examples

The compound of formula (I) as a free base compound has a quite strong lipophilic behavior (Log P 4.13). The presence of three main basic functional groups with calculated pKa of 8.9, 3.8, and 0.5 (MarvinSketch™ 18.24.0, ChemAxon Ltd.) makes it soluble in water at pH<1.8 but practically insoluble at pH≥4.5 with a solubility ≤0.02 mg/mL. Taking into account these characteristics, and based on pH requirements for intravenous (IV) solutions which is generally set ≥4.0, solubility studies were carried out to identify solubilizers able to substantially increase the aqueous solubility of the compound of formula (I).

### Methodologies

Solubility measurements were performed as follows: Two vials are prepared for solubility testing at 0 h (vial 1) and at 24 h (vial 2). The vial contents are prepared starting with 2.5 mg of the compound of formula (I). The primary excipient is added followed by any further excipient and then the buffered water to a total volume of 500 μL. All components are mixed in a vortex mixer for ca. 5 minutes. If everything is dissolved an additional 2.5 mg of the compound of formula (I) is added and vortexed again for ca. 5 minutes. This is repeated until a saturated solution (suspension) is obtained.

The solubility in vial 1 is measured immediately as described below. The components of vial 2 are stirred together in the vial with a magnetic stirrer for 24 hours and then vortexed for ca. 5 minutes prior to measuring solubility.

To measure solubility 400 μl of the suspension are taken and placed in a centrifugation device above a 0.2 μm filter. Centrifugation is performed at 14,000 rpm for 10 minutes and if necessary repeated for additional 10 minutes. The filter is removed from the vial and 100 μL is taken and diluted with 400 μL high-performance liquid chromatography (HPLC) diluent solution (see below) or diluted with 900 μL HPLC diluent solution in the event 5 mg or more of the compound of formula (I) was used. The pH of the solution is measured. A further 100 μL of the vial solution is taken and diluted with 400 μL of HPLC diluent solution or diluted with 900 μL HPLC diluent solution in the event 5 mg or more of the compound of formula (I) was used. The assay of the solution versus control stock solution is measured by HPLC and the solubility is calculated as described below. The HPLC diluent solution is 0.1% trifluoroacetic acid (TFA) in acetonitrile (ACN) and water (1:1 v/v).

The pH Meter used is calibrated on each day with standard buffers (pH) 2.00, 4.01, 7.00, 9.00 and the slope and offset values are determined. The specified acceptance criteria for the slope is 90-105% and the offset is-30 mV to 30 mV.

All solubility tests are performed at room temperature.

The HPLC method for assessing solubility is performed as follows:

| System | HPLC (>400 bar) | |
|---|---|---|
| Mobile phase A | 0.1% TFA in water | |
| Mobile phase B | 0.1% TFA in ACN | |
| Flow | 1.2 | mL/min |
| Column | Waters XBridge ™ C8, | |
| | 3.5 μm, 4.6 × 150 mm | |
| Column oven | 35° | C. |
| Injection volume | 5 | μL |
| Autosampler temperature | 20° | C. |
| Wavelength | 242 | nm |

TABLE 1
HPLC gradient

| | Mobile | Mobile |
|---|---|---|
| Time | Phase | Phase |
| [min] | A [%] | B [%] |
| 0 | 95 | 5 |
| 5.0 | 95 | 5 |
| 10.0 | 80 | 20 |
| 28.0 | 50 | 50 |
| 32.0 | 5 | 95 |
| 35.0 | 5 | 95 |
| 36.0 | 95 | 5 |
| 40.0 | 95 | 5 |

The solubility of the compound of formula (I) is calculated according to the following formula:

$$Solubility ( mg / mL ) = \frac{A - b}{a} * n$$

Solubility=solubility of compound of formula (I) (mg/mL)
A=peak area of the compound of the sample tested
a=value of constant “a” in Linear regression equation y=ax+b
b=value of constant “b” in Linear regression equation y=ax+b
n=dilution time
Formulations for stability assessment are prepared as follows:

500 mL batches of each formulation (target pH 4.0 and pH 5.5) are prepared according to the recipe in Table 2 below. The vials used were 50 mL 20 mm Crown, type I, Vial (Gerresheimer), part number 80018303030. The stoppers used were 20 mm S10-F451 Stopper 4432/50 Gray, (Flurotec™ On Plug Only) B2-40 Coating Westar RS part number 19700021.

Each formulation is filtered with a 0.22 μm nylon syringe filter. Vials are filled with 10 mL of formulation, stoppered, sealed and placed at 40° C./75% relative humidity (RH) or at 60° C. (uncontrolled humidity) for up to 14 days. Each vial is placed inverted so that maximum stopper contact with the solution during the stability conditions can be achieved. At each time-point, vials from both conditions and formulations are pulled and analyzed for assay and impurities.

TABLE 2
Formulation recipes for stability testing

| | Formulation pH 4.0 | | Formulation pH 5.5 | |
|---|---|---|---|---|
| | | Amount | | Amount |
| | | required | | required |
| | | for 500 mL | | for 500 mL |
| Component | mg/mL | batch (g) | mg/mL | batch (g) |
| Compound of | 2.00 | 1.00 | 2.00 | 1.00 |
| formula (I) | | | | |
| Hydrochloric | 3.94 | 1.97 | 3.94 | 1.97 |
| Acid | | | | |
| (concentrated) | | | | |
| Citric Acid, | 6.30 | 3.15 | 6.30 | 3.15 |
| Monohydrate | | | | |
| Sodium | Adjust to | Adjust to | Adjust to | Adjust to |
| Hydroxide | pH 4.0 | pH 4.0 | pH 5.5 | pH 5.5 |
| SBE-beta-CD | 40.00 | 20.00 | 40.00 | 20.00 |
| Sodium Chloride | 3.00 | 1.50 | 3.00 | 1.50 |
| Water for | q.s. | q.s to | q.s | q.s to |
| Injection | | 500 mL | | 500 mL |

The assay and impurities as measured using the same HPLC method as used for assessing solubility.

### Results

### Effect of Single Solubilizers

Various potential solubilizers were tested for their ability to solubilize the compound of formula (I), as shown in Table 3.

TABLE 3
Effect of excipients on the solubility of the compound of formula (I)

| Vehicle, | | 0 h test result | | 24 h test result | | |
|---|---|---|---|---|---|---|
| target pH | Excipient | pHₑₓₚₑᵣᵢₘₑₙₜₐₗ | Solubility(mg/mL) | pHₑₓₚₑᵣᵢₘₑₙₜₐₗ | Solubility(mg/mL) | |
| CBS 30 | — | 4.0 | 0.16 | 4.2 | 0.12 | |
| mM, pH | | | | | | |
| 4.0 | | | | | | |
| CBS 30 | PEG 400 | 4.1 | 0.22 | 4.3 | 0.15 | |
| mM, pH | 20 mg/ | | | | | |
| 4.0 | mL | | | | | |
| | PEG 400 | 4.1 | 0.34 | 4.4 | 0.23 | |
| | 50 mg/ | | | | | |
| | mL | | | | | |
| | PEG 100 | 4.3 | 0.60 | 4.5 | 0.40 | |
| | mg/mL | | | | | |
| | PEG 400 | 6.3 | 4.10 | 6.4 | ≥4.55 | |
| | 500 mg/ | | | | | |
| | mL | | | | | |
| CBS 30 | PG 50 | 4.1 | 0.20 | 4.3 | 0.11 | |
| mM, pH | mg/mL | | | | | |
| 4.0 | PG 100 | 4.1 | 0.28 | 4.4 | 0.15 | |
| | mg/mL | | | | | |
| | PG 500 | 5.4 | 3.74 | 5.3 | 2.20 | |
| | mg/mL | | | | | |
| CBS 30 | Ethanol | 4.1 | 0.16 | 4.2 | 0.11 | |
| mM, pH | 20 mg/ | | | | | |
| 4.0 | mL | | | | | |
| | Ethanol | 4.2 | 0.19 | 4.3 | 0.09 | |
| | 50 mg/ | | | | | |
| | mL | | | | | |
| | Ethanol | 4.2 | 0.26 | 4.4 | 0.14 | |
| | 100 mg/ | | | | | |
| | mL | | | | | |
| CBS 30 | Glycerol | 4.1 | | 0.18 | 4.3 | 0.12 |
| mM, pH | 50 mg/ | | | | | |
| 4.0 | mL | | | | | |
| | Glycerol | 4.1 | | 0.21 | 4.3 | 0.13 |
| | 100 mg/ | | | | | |
| | mL | | | | | |
| | Glycerol | 4.6 | | 1.06 | 4.8 | 0.26 |
| | 500 mg/ | | | | | |
| | mL | | | | | |
| CBS 30 | SBE-beta- | 4.1 | | 0.89 | 4.2 | 1.11 |
| mM, pH | CD 10 | | | | | |
| 4.0NaCl | mg/mL | | | | | |
| 3 mg/mL | SBE-beta- | 4.1 | | 1.84 | 4.2 | 2.54 |
| | CD 25 | | | | | |
| | mg/mL | | | | | |
| | SBE-beta- | 4.0 | | 2.48 | 4.2 | 3.80 |
| | CD 40 | | | | | |
| | mg/mL | | | | | |
| CBS 30 | HP-beta- | 4.10 | | 1.67 | 4.28 | 0.98 |
| mM, pH | CD 40 | | | | | |
| 4.0NaCl | mg/mL | | | | | |
| 3 mg/mL | | | | | | |

CBS = Citrate Buffer Solution - PG = Propylene Glycol - SBE-beta-CD = Sulfobutyl ether beta-Cyclodextrin - HP-beta-CD - = hydroxypropyl beta-cyclodextrin

High solubility of the compound of formula (I) at concentrations of solubilizer suitable for intravenous use was observed with sulfobutyl ether beta-cyclodextrin (SBE-beta-CD). Low solubility of the compound of formula (I) was observed with other solubilizers tested at concentrations suitable for intravenous, including hydroxypropyl beta-cyclodextrin. The concentrations of PEG 400 and propylene glycol resulting in higher solubility of the compound of formula (I) were 500 mg/mL, which is too high for intravenous use.

Effect of pH on Solubility with SBE-Beta-CD

The effect of pH on the solubility of the compound of formula (I) with SBE-beta-CD was investigated. The results are shown in Table 4.

TABLE 4
Effect of pH variation on the solubility of the compound of formula (I)

| | | 0 h test result | | | |
|---|---|---|---|---|---|
| Vehicle, | | | Solubility | 24 h test result | |
| target pH | Excipient | pHₑₓₚₑᵣᵢₘₑₙₜₐₗ | (mg/mL) | pHₑₓₚₑᵣᵢₘₑₙₜₐₗ | Solubility(mg/mL) |
| CBS 30 mM, | SBE-beta-CD | 2.2 | 6.59 | 2.2 | 5.47 |
| pH 2.0NaCl | 40 mg/mL | | | | |
| 3 mg/mL | | | | | |
| CBS 30 mM, | | 4.0 | 2.48 | 4.2 | 3.80 |
| pH 4.0NaCl | | | | | |
| 3 mg/mL | | | | | |
| CBS 30 mM, | | 5.0 | 2.24 | 5.3 | 3.52 |
| pH 5.0NaCl | | | | | |
| 3 mg/mL | | | | | |
| CBS 30 mM, | | 5.5 | 1.63 | 5.7 | 3.10 |
| pH 5.5NaCl | | | | | |
| 3 mg/mL | | | | | |
| CBS 30 mM, | | 7.0 | 2.23 | 7.0 | 2.23 |
| pH 7.0NaCl | | | | | |
| 3 mg/mL | | | | | |

CBS = Citrate Buffer Solution - SBE-beta-CD = Sulfobutyl ether beta-Cyclodextrin

It was observed that solution pH affects solubility, with lower solubility of the compound of formula (I) observed at higher pH. High solubility was observed in particular between pH 4 to pH 5.5.

### Effect of Additional Excipients on Solubility

The effect of adding additional excipients on the solubility of the compound of formula (I) with SBE-beta-CD was investigated. The results are shown in Table 5.

TABLE 5
Effect of additional excipients on the solubility of the compound of formula (I)

| Vehicle, | Excipient | Excipient | 0 h test result | | 24 h test result | |
|---|---|---|---|---|---|---|
| target pH | 1 | 2 | pHₑₓₚₑᵣᵢₘₑₙₜₐₗ | Solubility(mg/mL) | pHₑₓₚₑᵣᵢₘₑₙₜₐₗ | Solubility(mg/mL) |
| CBS 30 | SBE-beta- | PEG | 4.1 | 2.48 | 4.2 | 3.73 |
| mMpH | CD 40 | 4001 mg/ | | | | |
| 4.0NaCl | mg/mL | mL | | | | |
| 3 mg/mL | | PEG | 4.1 | 2.34 | 4.2 | 3.75 |
| | | 4005 mg/ | | | | |
| | | mL | | | | |
| | | PEG | 4.1 | 2.59 | 4.2 | 3.79 |
| | | 40010 | | | | |
| | | mg/mL | | | | |
| | | PEG | 4.1 | 2.57 | 4.2 | 3.81 |
| | | 40020 | | | | |
| | | mg/mL | | | | |
| | | PEG | 4.1 | 2.67 | 4.2 | 3.86 |
| | | 40030 | | | | |
| | | mg/mL | | | | |
| CBS 30 | SBE-beta- | PEG | 4.2 | 2.81 | 4.3 | 4.06 |
| mMpH 4.0 | CD 56 | 40022 | | | | |
| | mg/mL | mg/mL | | | | |
| CBS 30 | SBE-beta- | Glycerol: | 4.1 | 2.40 | 4.3 | 3.72 |
| mMpH | CD 40 | 1 mg/mL | | | | |
| 4.0NaCl | mg/mL | Glycerol: | 4.2 | 2.32 | 4.3 | 3.71 |
| 3 mg/mL | | 5 mg/mL | | | | |
| | | Glycerol | 4.1 | 2.33 | 4.2 | 3.57 |
| | | 50 mg/ | | | | |
| | | mL | | | | |
| CBS 30 | SBE-beta- | Propylene | 4.1 | 2.36 | 4.3 | 3.82 |
| mMpH | CD 40 | glycol: 5 | | | | |
| 4.0NaCl | mg/mL | mg/mL | | | | |
| 6 mg/mL | | Propylene | 4.1 | 2.42 | 4.2 | 3.65 |
| | | glycol: 10 | | | | |
| | | mg/mL | | | | |
| | | Propylene | 4.1 | 2.31 | 4.2 | 3.44 |
| | | glycol: 20 | | | | |
| | | mg/mL | | | | |
| | | Propylene | 4.1 | 2.15 | 4.2 | 3.35 |
| | | glycol: 30 | | | | |
| | | mg/mL | | | | |
| CBS 30 | SBE-beta- | PEG | 4.1 | 2.42 | 4.2 | 3.67 |
| mMpH | CD 40 | 40005 | | | | |
| 4.0NaCl | mg/mL | mg/mL | | | | |
| 3 mg/mL | | | | | | |
| CBS 30 | SBE-beta- | Ethanol16 | 4.1 | 2.49 | 4.3 | 3.85 |
| mMpH 4.0 | CD 54 | mg/mL | | | | |
| | mg/mL | | | | | |
| CBS 30 | SBE-beta- | PEG | 4.2 | 2.89 | 4.3 | 4.03 |
| mMpH 4.0 | CD 54 | 30056 | | | | |
| | mg/mL | mg/mL | | | | |
| CBS 30 | SBE-beta- | Kollidon ™12 | 4.1 | 2.37 | 4.3 | 3.69 |
| mMpH | CD 40 | PF: 2.5 | | | | |
| 4.0NaCl | mg/mL | mg/mL | | | | |
| 6 mg/mL | | Kollidon ™12 | 4.0 | 2.51 | 4.2 | 3.75 |
| | | PF: 5 | | | | |
| | | mg/mL | | | | |
| | | Kollidon ™12 | 4.1 | 2.52 | 4.2 | 3.70 |
| | | PF: 10 | | | | |
| | | mg/mL | | | | |
| CBS 30 | SBE-beta- | Kollidon ™12 | 4.1 | 2.59 | 4.2 | 3.71 |
| mMpH | CD40 | P1885 | | | | |
| 4.0NaCl | mg/mL | mg/mL | | | | |
| 3 mg/mL | | | | | | |
| CBS 30 | SBE-beta- | Soybean | 4.1 | 2.52 | 4.3 | 3.60 |
| mMpH | CD 40 | lecithin 10 | | | | |
| 4.0NaCl | mg/mL | mg/mL | | | | |
| 3 mg/mL | | | | | | |
| CBS 30 | SBE-beta- | HPMC5 | 4.1 | 2.23 | 4.3 | 3.53 |
| mMpH | CD 40 | mg/mL | | | | |
| 4.0NaCl | mg/mL | | | | | |
| 3 mg/mL | | | | | | |
| CBS 30 | SBE-beta- | Tween ™ | 4.1 | 2.95 | 4.2 | 4.04 |
| mMpH | CD 40 | 805 mg/ | | | | |
| 4.0NaCl | mg/mL | mL | | | | |
| 3 mg/mL | | | | | | |
| CBS 30 | SBE-beta- | Na | 4.1 | 1.71 | n. a. | 2.67 |
| mMpH | CD 40 | CMC5 | | | | |
| 4.0NaCl | mg/mL | mg/mL | | | | |
| 3 mg/mL | | | | | | |

CBS = Citrate Buffer Solution - SBE-beta-CD = Sulfobutyl ether beta-Cyclodextrin - HPMC = Hydroxypropyl-methyl-cellulose - Na CMC = Sodium Carboxymethyl-Cellulose Sodium

It was observed that PEG 400, glycerol, propylene glycol and PEG 4000 do not increase the solubility of the compound of formula (I) with SBE-beta-CD. Solubilization with ethanol and PEG 300 is similar to PEG 400 (note that a higher concentration of SBE-beta-CD was used in these trials). Kollidon™ 12 PF, Kolliphor™ P188, soybean lecithin have a very small impact on solubility with SBE-beta-CD. The addition of Tween™ 80 slightly improves solubility. The addition of carboxymethylcellulose sodium decreases solubility and the solution became sticky at 24 hours. These results also show that saline has a very small impact on solubility.

### Effect of Buffer on Solubility

The effect of different buffers on the solubility of the compound of formula (I) with SBE-beta-CD was investigated. The results are shown in Table 6.

TABLE 6
Effect of buffer salt on the solubility of the compound of formula (I)

| Vehicle, | | 0 h test result | | 24 h test result | |
|---|---|---|---|---|---|
| target pH | Excipient | pHₑₓₚₑᵣᵢₘₑₙₜₐₗ | Solubility(mg/mL) | pHₑₓₚₑᵣᵢₘₑₙₜₐₗ | Solubility(mg/mL) |
| CBS 30 mM pH | SBE-beta-CD | 4.0 | 2.48 | 4.2 | 3.80 |
| 4.0NaCl 3 mg/mL | 40 mg/mL | | | | |
| ABS 30 mMpH | | 4.0 | 2.88 | 4.6 | 3.72 |
| 4.0NaCl 3 mg/mL | | | | | |
| GBS 30 mMpH | | 4.1 | 2.59 | 4.5 | 3.94 |
| 4.0NaCl 3 mg/mL | | | | | |
| LBS 30 mMpH | | 4.1 | 2.69 | 4.3 | 3.98 |
| 4.0NaCl 3 mg/mL | | | | | |
| ASBS 30 mMpH | | 4.0 | 2.88 | 4.6 | 3.72 |
| 4.0NaCl 3 mg/mL | | | | | |

CBS = Citrate Buffer Solution - ABS = Acetate Buffer Solution - GBS = Gluconate Buffer Solution - LBS = Lactate Buffer Solution - ASBS = Aspartate Buffer Solution - SBE-beta-CD = Sulfobutyl ether beta-Cyclodextrin

It was observed that the solubility of the compound of formula (I) with the buffers tested (citrate acid, acetic acid, gluconic acid, lactic acid and aspartic acid buffer) was approximately the same.

### Effect of pH on Stability

The effect of pH on formulation stability of the compound of formula (I) with SBE-beta-CD was tested. The results are shown in Table 7.

TABLE 7
Effect of pH on formulation stability with SBE-beta-CD

| | | | Assay (% of | |
|---|---|---|---|---|
| | | | theoretical | |
| | | | value of | Total |
| Target pH, | Time | | compound of | impurities |
| temperature | point | pHₑₓₚₑᵣᵢₘₑₙₜₐₗ | formula (I)) | (% area) |
| pH 4.040° C. | TO | 4.1 | 96.8 | 1.1 |
| | Day 1 | 4.1 | 98.1 | 1.1 |
| | Day 3 | 4.0 | 98.7 | 1.3 |
| | Day 7 | 4.1 | 103.1 | 1.3 |
| | Day 14 | 4.1 | 97.7 | 1.4 |
| pH 4.060° C. | TO | 4.1 | 96.8 | 1.1 |
| | Day 1 | 4.1 | 97.9 | 1.4 |
| | Day 3 | 4.1 | 98.3 | 2.0 |
| | Day 7 | 4.1 | 97.9 | 3.4 |
| | Day 14 | 4.1 | 87.0 | 4.7 |
| pH 5.540° C. | TO | 5.6 | 99.0 | 1.1 |
| | Day 1 | 5.6 | 97.9 | 1.2 |
| | Day 3 | 5.6 | 100.6 | 1.3 |
| | Day 7 | 5.7 | 100.1 | 1.2 |
| | Day 14 | 5.6 | 94.7 | 1.3 |
| pH 5.560° C. | TO | 5.6 | 99.0 | 1.1 |
| | Day 1 | 5.6 | 97.9 | 1.3 |
| | Day 3 | 5.6 | 100.6 | 1.5 |
| | Day 7 | 5.7 | 100.1 | 2.0 |
| | Day 14 | 5.7 | 97.7 | 3.0 |

TO = time zero

The results show that the stability of formulation containing the compound of formula (I) with SBE-beta-CD is similar at pH 4.0 and pH 5.5 at 40° C./75% RH stressed conditions. However, at 60° C. stressed conditions the formulation at pH 5.5 had lower impurities and lower loss of assay compared to the formulation at pH 4.0.

1. A pharmaceutical composition comprising a compound of formula (I) 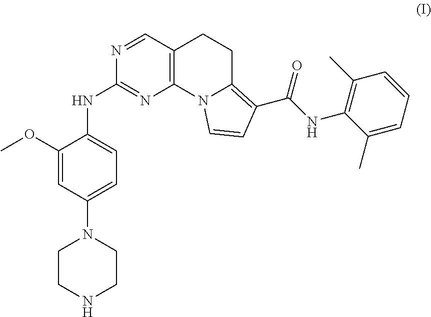 [SMILES: COc1cc(N2CCNCC2)ccc1Nc1ncc2c(n1)-n1ccc(C(=O)Nc3c(C)cccc3C)c1CC2]
or a pharmaceutically acceptable salt thereof and a modified beta-cyclodextrin.

2. The pharmaceutical composition of claim 1, wherein the modified beta-cyclodextrin is sulfobutyl ether beta-cyclodextrin (SBE-beta-CD).

3. The pharmaceutical composition of claim 2, wherein the SBE-beta-CD has a degree of substitution of about 6 to about 7.5.

4. The pharmaceutical composition of claim 2, wherein the SBE-beta-CD has an average degree of substitution of about 6 to about 7.5.

5. The pharmaceutical composition of claim 2, wherein the SBE-beta-CD has an average degree of substitution of about 6.5.

6. The pharmaceutical composition of claim 2, wherein the concentration of SBE-beta-CD in the pharmaceutical composition is up to about 400 mg/mL.

7. The pharmaceutical composition of claim 2, wherein the concentration of SBE-beta-CD in the pharmaceutical composition is about 10 mg/mL to about 100 mg/mL.

8. The pharmaceutical composition of claim 2, wherein the concentration of SBE-beta-CD in the pharmaceutical composition is about 30 mg/mL to about 50 mg/mL.

9. The pharmaceutical composition of claim 1, wherein the concentration of the compound of formula (I) or a pharmaceutically acceptable salt thereof in the pharmaceutical composition is up to about 3.5 mg/mL based on the compound of formula (I) as free base.

10. The pharmaceutical composition of claim 1, wherein the concentration of the compound of formula (I) or a pharmaceutically acceptable salt thereof is about 1.5 mg/mL to about 2.5 mg/ml based on the compound of formula (I) as free base.

11. The pharmaceutical composition of claim 1, wherein the concentration of the compound of formula (I) or a pharmaceutically acceptable salt thereof is about 2 mg/mL based on the compound of formula (I) as free base.

12. The pharmaceutical composition of claim 1, wherein the pH of the pharmaceutical composition is about 3.5 to about 7.

13. The pharmaceutical composition of claim 1, wherein the pH of the pharmaceutical composition is about 4 to about 6.5.

14. The pharmaceutical composition of claim 1, wherein the pH of the pharmaceutical composition is 4.5 to 6.5.

15. The pharmaceutical composition of claim 1, wherein the pharmaceutical composition comprises a salt as osmolality agent.

16. The pharmaceutical composition of claim 15, wherein the salt is sodium chloride.

17. The pharmaceutical composition of claim 15, wherein the salt concentration in the pharmaceutical composition is up to about 10 mg/mL.

18. The pharmaceutical composition of claim 15, wherein the salt concentration in the pharmaceutical composition is about 0.5 mg/ml to about 8 mg/mL.

19. The pharmaceutical composition of claim 15, wherein the salt concentration in the pharmaceutical composition is about 3 mg/mL.

20. The pharmaceutical composition of claim 1, wherein the pharmaceutical composition comprises the compound of formula (I) or a pharmaceutically acceptable salt thereof at a concentration of about 1 mg/mL to about 3 mg/mL based on the compound of formula (I) as free base, and SBE-beta-CD having an average degree of substitution of about 6 to about 7.5 at a concentration of about 10 mg/mL to about 100 mg/mL, and wherein the pH of the pharmaceutical composition is about 4 to about 6.5.

21. The pharmaceutical composition of claim 1, wherein the pharmaceutical composition consists essentially of the compound of formula (I) as free base, SBE-beta-CD having an average degree of substitution of about 6 to about 7.5, one or more buffering agents, optionally a salt, and optionally water, and wherein the pH of the pharmaceutical composition is about 4 to about 6.5.

22. The pharmaceutical composition of claim 1, wherein the pharmaceutical composition consists essentially of the compound of formula (I) as free base, at a concentration of about 1 mg/mL to about 3 mg/mL, SBE-beta-CD having an average degree of substitution of about 6 to about 7.5 at a concentration of about 10 mg/mL to about 100 mg/mL, one or more buffering agents, optionally a salt, and optionally water, and wherein the pH of the pharmaceutical composition is about 4 to about 6.5.

23. The pharmaceutical composition of claim 1, wherein the pharmaceutical composition consists essentially of the compound of formula (I) as free base at a concentration of about 1.5 mg/mL to about 2.5 mg/mL, SBE-beta-CD having an average degree of substitution of about 6 to about 7.5 at a concentration of about 30 mg/mL to about 50 mg/mL, one or more buffering agents, optionally a salt, and optionally water, and wherein the pH of the pharmaceutical composition is about 4 to about 6.5.

24. The pharmaceutical composition of claim 1, wherein the pharmaceutical composition is one wherein the components and the quantities of each component per mL of pharmaceutical composition are as follows:

| Component | Quantity |
|---|---|
| compound of formula (I) as free base | about 2 mg |
| SBE-β-CD, average degree of | about 40 mg |
| substitution of about 6 to about 7.5 | |
| citrate buffer | about 30 mM |
| sodium chloride | about 3 mg/mL |
| sodium hydroxide | quantity sufficient to adjust |
| | the pH to 4.5 to 6.5 |
| hydrochloric acid | quantity sufficient to adjust |
| | the pH to 4.5 to 6.5 |
| water for injection | q.s. to 1 mL |
| Total Volume | 1 mL |

25. The pharmaceutical composition of claim 1, wherein the compound of formula (I) or a pharmaceutically acceptable salt thereof is the compound of formula (I) as free base.

26. The pharmaceutical composition of claim 1, wherein term “about” means a variation of no more than 5% of the relevant figure.

27. The pharmaceutical composition of claim 1, wherein the pharmaceutical composition is suitable for intravenous administration.

28. A pharmaceutical composition as defined in claim 1, for use in treating a neoplastic disease in a patient.

29. (canceled)

30. A method of treating a neoplastic disease in a patient in need thereof comprising:
administering a pharmaceutical composition as defined in claim 1 in a therapeutically effective amount to said patient.

31. (canceled)

32. The method of claim 30, wherein the neoplastic disease is a cancer selected from breast cancer, gastric cancer, colorectal cancer, liver cancer, endometrial cancer, ovarian cancer, esophageal cancer, lung cancer, Kaposi's sarcoma, cervical cancer, pancreatic cancer, melanoma, prostate cancer, bladder cancer, and leukemia.
